(19) United States
(12) Patent Application Publication
(10) Pub. No.: US 2026/0183499 A1
(43) Pub. Date: Jul. 2, 2026

(54) VENTILATOR MANIFOLD ASSEMBLY

(71) Applicant: HILL-ROM SERVICES PTE. LTD., OT (SG)

(72) Inventors: Mohammad Sahlabadi, Irvine, CA (US); Joshua Cruz, Yorba Linda, CA (US)

(21) Appl. No.: 19/227,041

(22) Filed: Jun. 3, 2025

Related U.S. Application Data
(60) Provisional application No. 63/655,903, filed on Jun. 4, 2024.

(51) Int. Cl. A61M 16/00

(52) U.S. Cl. CPC A61M 16/0003; A61M 2016/0027; A61M 2205/3331

(57) ABSTRACT

A manifold body for a ventilator includes a first interior region defining a flow passage extending from a flow input of the manifold body to a flow output of the manifold body and a second interior region defining a pressure sense line for providing fluid communication between the flow passage and a pressure sense output of the manifold body. The pressure sense line may have a first cross-sectional area at a position where the pressure sense line opens into the flow passage and a second cross-sectional area greater than the first cross-sectional area at a position nearer to the pressure sense output. The ventilator may have a pressure relief valve and a gas inlet connector with low pressure drop.

### CROSS-REFERENCE TO RELATED APPLICATIONS

The present application claims priority to U.S. Provisional Application Ser. No. 63/655,903 filed Jun. 4, 2024, the disclosure of which is incorporated herein by reference.

### STATEMENT RE: FEDERALLY SPONSORED RESEARCH/DEVELOPMENT

Not Applicable

### BACKGROUND

The present disclosure relates generally to ventilation therapy and, more particularly, to a manifold assembly of a ventilator.

A wide range of clinical conditions may require some form of ventilation therapy, whereby the patient's work of breathing is assisted by the flow of pressurized gas from a ventilator to the patient's airway. These conditions may include hypoxemia, various forms of respiratory insufficiency, and airway disorders. There are also non-respiratory and non-airway diseases that require ventilation therapy, such as congestive heart failure and neuromuscular diseases.

To improve the quality of life of many patients who require long-term ventilation therapy, ventilation systems have been developed which are miniaturized and portable. Some of these systems, for example, the Life2000® system by Breathe Technologies, Inc., are so lightweight and compact that in their extended range or stand-alone configurations, they are wearable by the patient. These systems make use of a source of pressurized ventilation gas to operate. In the stationary or extended-range configuration, the source of pressurized gas may be a stationary compressor unit, which may be kept in a patient's home. In the stand-alone configuration, which may be generally used when the patient is outside the home, the portable, wearable ventilator generally receives its ventilation gas from a pressurized gas cylinder or a portable compressor.

Many of the above clinical conditions and other clinical conditions may also require or benefit from supplemental oxygen therapy, whereby the gas introduced to the patient's airway is augmented by the presence of additional oxygen such that the patient inspires gas having oxygen levels above atmospheric concentration (20.9% at 0% humidity). Supplemental oxygen therapy involves the patient receiving supplemental oxygen gas from an oxygen gas source, which is typically a compressed or cryogenic oxygen cylinder, or an oxygen gas generator. For many years, patients who wished to be mobile relied on oxygen cylinders. However, in recent years, miniaturization and improvements in battery technology has resulted in the development of portable oxygen concentrators.

Portable oxygen concentrators typically operate by pressure swing adsorption (PSA), in which ambient air is pressurized by a compressor and passed through an adsorbent sieve bed. The sieve bed is typically formed of a zeolite which preferentially adsorbs nitrogen when at high pressure while oxygen passes through. Once the sieve bed reaches its capacity to adsorb nitrogen, the pressure can be reduced. This reduction in pressure causes the adsorbed nitrogen to be desorbed so it can be purged, leaving a regenerated sieve bed that is again ready to adsorb nitrogen. With repeated cycles of this operation, an enriched oxygen gas may be generated. Typically, portable oxygen concentrators have at least two sieve beds so that one may operate while the other is being purged of the nitrogen and vented. Typical portable oxygen concentrators today output an enriched oxygen gas with a purity of around 87-96% oxygen. Among existing oxygen concentrators today which may be considered portable (especially by an individual suffering from a respiratory condition), there are generally two types available. The first type, which is larger and heavier, is usually capable of continuous flow delivery. Models of this type typically weigh between 5-10 kg, have maximum flow rates of around 5-6 liters per minute or less, and are generally configured with wheels and a handle, often mimicking the appearance of a suitcase. The second type are lighter units more suitable for being carried or worn in a satchel, handbag, or a backpack. Models of this type typically weigh less than 2.5 kg and are usually limited to pulsed delivery modes with maximum flow rates of around 2 liters per minute or less.

Portable oxygen concentrators have a substantial cost and convenience advantage over pressurized oxygen cylinders, due to the pressurized oxygen cylinders requiring ongoing refilling or replacement. Additionally, portable oxygen concentrators are considered to be significantly safer than pressurized oxygen cylinders. This safety consideration can have a substantial impact on a patient's quality of life because many portable oxygen concentrators have been approved by the FAA for use by travelers on commercial airlines, whereas oxygen cylinders are universally banned on commercial flights. Consequently, patients with pressurized oxygen cylinders must make expensive and time-consuming preparations with an airline ahead of time or forego airline travel entirely.

For patients with conditions where assistance with the work of breathing is not required, supplemental oxygen therapy alone, without ventilation therapy, may be sufficient. However, for many patients, combined ventilation therapy and supplemental oxygen therapy may be a more optimal treatment. In healthy patients, sufficient ventilation to perform the work of breathing may typically require minute ventilation rates of between 5 and 8 L/min while stationary, which may double during light exercise, and which may exceed 40 L/min during heavy exercise. Patients suffering from respiratory conditions may require substantially higher rates, and substantially higher instantaneous rates. This is especially true when these patients are outside the home and require portability, as at these times such patients are often also involved in light exercise.

It may thus be seen that patients who would prefer to receive this combined mode of treatment are substantially limited, since in many cases existing portable oxygen concentrators do not output gas at pressures and/or volumes high enough to be used with a wearable, portable ventilator without the presence of an additional source of compressed gas. While existing systems and methods that seek to provide a combined supplemental oxygen/ventilation system have been developed in the prior art, these existing systems suffer from various deficiencies which Applicant has addressed in the system described in its U.S. Pat. No. 11,607,519 entitled O2 CONCENTRATOR WITH SIEVE BED BYPASS AND CONTROL METHOD THEREOF, the disclosure of which is incorporated herein by reference.

In the case of ventilation therapy and combined ventilation therapy with supplemental oxygen, the delivery of gas to the patient via a patient interface (e.g., nasal pillows, mask, etc.) typically involves the passage of gas through a ventilator manifold. Within the manifold, the flow of gas interfaces with one or more relief valves and pressure taps for pressure and/or flow sensing, as well as a main solenoid valve that controls the timing and volume of gas delivered. All of these elements, as well as the connection mechanism for introducing the gas to the manifold from the gas source, inevitably cause unwanted pressure drops, limiting the performance of the ventilator. Moreover, moisture accumulation in pressure taps may require periodic drying in order to prevent damage to sensitive electronic components. In particular, with conventional pressure taps, capillary action causes any moisture that accumulates due to rainout in the manifold to work its way through the narrow pressure sense line toward the pressure sensor. In order to prevent this moisture from reaching the pressure sensor and potentially causing the ventilator to fail, there must typically be some procedure for drying out accumulated moisture, which is time-consuming and may entail additional mechanical considerations such as a means to conveniently separate the pressure sensor from the pressure sense line.

### BRIEF SUMMARY

The present disclosure contemplates various structures and methods for overcoming the above drawbacks accompanying the related art. One aspect of the embodiments of the present disclosure is a manifold body for a ventilator. The manifold body may comprise a first interior region defining a flow passage extending from a flow input of the manifold body to a flow output of the manifold body. The manifold body may further comprise a second interior region defining a pressure sense line for providing fluid communication between the flow passage and a pressure sense output of the manifold body. The pressure sense line may have a first cross-sectional area at a position where the pressure sense line opens into the flow passage and a second cross-sectional area greater than the first cross-sectional area at a position nearer to the pressure sense output.

The pressure sense line may comprise a conical void whose cross-sectional area increases in a direction from the flow passage to the pressure sense output. The first interior region may further define an opening in the flow passage between the flow input and the flow output of the manifold body. The opening may be configured for arrangement of a pressure relief valve in parallel to the flow passage.

Another aspect of the embodiments of the present disclosure is a ventilator comprising a flow passage for compressed gas and a pressure sense line for providing fluid communication between the flow passage and a pressure sensor. The pressure sense line may have a first cross-sectional area at a position where the pressure sense line opens into the flow passage and a second cross-sectional area greater than the first cross-sectional area at a position nearer to the pressure sensor.

The pressure sense line may comprise a conical void whose cross-sectional area increases in a direction from the flow passage to the pressure sensor. The ventilator may comprise the pressure sensor. The ventilator may comprise a pressure sense passage connectable to a pressure sense lumen of a patient circuit. The ventilator may comprise a second pressure sense line for providing fluid communication between the pressure sense passage and a second pressure sensor. The second pressure sense line may have a first cross-sectional area at a position where the second pressure sense line opens into the pressure sense passage and a second cross-sectional area greater than the first cross-sectional area at a position nearer to the second pressure sensor. The ventilator may comprise a purge valve operable to purge the pressure sense lumen of the patient circuit via the pressure sense passage. The purge valve may be operable to purge the pressure sense lumen by fluidly connecting the pressure sense lumen of the patient circuit to the flow passage via the pressure sense passage. The ventilator may comprise an autozero valve operable to zero the second pressure sensor simultaneously with operation of the purge valve.

Another aspect of the embodiments of the present disclosure is a ventilator comprising a flow passage for compressed gas and a pressure relief valve arranged in parallel to the flow passage and configured to dump compressed gas from the flow passage.

The pressure relief valve may be fluidly coupled to an opening in the flow passage located at a ninety-degree turn of the flow passage. The pressure relief valve may include a seat fluidly coupled to an opening in the flow passage and defining a dump passage for dumping the compressed gas. The pressure relief valve may include a diaphragm configured to open and close the dump passage. The seat may further define a main passage that is fluidly coupled to the opening in the flow passage. The dump passage may be defined concentrically around the main passage.

Another aspect of the embodiments of the present disclosure is a ventilator comprising a flow passage for compressed gas and a gas inlet connector connectable to the flow passage. The gas inlet connector may include a connector housing defining a bore with a first portion having a first diameter, a second portion having a second diameter less than the first diameter, and a shoulder between the first and second portions. The gas inlet connector may further include a valve disposed within the bore. The valve may have a first end that has a conical shape and a second end that defines a surface configured to seat against the shoulder. The valve may further have one or more alignment tabs that protrude from the second end into the second portion of the bore. The gas inlet connector may further include a spring biasing the valve away from the shoulder.

The one or more alignment tabs may comprise three radially configured alignment tabs. The three radially configured alignment tabs may be evenly spaced in a circumferential direction of the second portion of the bore.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The above and other features and advantages of the various embodiments disclosed herein will be better understood with respect to the following description and drawings, in which like numbers refer to like parts throughout, and in which:

FIG. 1 is a schematic diagram of a ventilator manifold assembly according to an embodiment of the present disclosure;

FIG. 2 is a top perspective view of the ventilator manifold assembly including a manifold body and related components;

FIG. 3 is a top view of the ventilator manifold assembly;

FIG. 4A is a schematic diagram of the ventilator manifold assembly emphasizing a flow passage thereof;

FIG. 4B is a top perspective view of the manifold body emphasizing the flow passage;

FIG. 5A is a schematic diagram of the ventilator manifold assembly emphasizing Ps, dP, and Pd pressure sense lines thereof;

FIG. 5B is a top perspective view of the manifold body emphasizing the Ps, dP, and Pd pressure sense lines;

FIG. 6A is a schematic diagram of the ventilator manifold assembly emphasizing an airway pressure sense line Paw thereof;

FIG. 6B is a top perspective view of the manifold body emphasizing the airway pressure sense line Paw;

FIG. 7A is a schematic diagram of the ventilator manifold assembly emphasizing a purge line thereof;

FIG. 7B is a top perspective view of the manifold body emphasizing the purge line;

FIG. 8 is top perspective view of the manifold body;

FIG. 9 is a top side perspective view of the manifold body;

FIG. 10 is a bottom side perspective view of the manifold body;

FIG. 11 is a side view of the manifold body;

FIG. 12 shows a simulation of gas flow within the manifold body when a pressure relief valve is closed;

FIG. 13 shows a simulation of gas flow within the manifold body when the pressure relief valve is open;

FIG. 14 is an exploded perspective view of the pressure relief valve;

FIG. 15A is a schematic diagram of the ventilator manifold assembly emphasizing passages associated with the pressure relief valve;

FIG. 15B is a top perspective view of the manifold body emphasizing the passages associated with the pressure relief valve;

FIG. 15C is a bottom perspective view of the manifold body emphasizing the passages associated with the pressure relief valve;

FIG. 16 is a cross-sectional view of a gas inlet connector of the ventilator manifold assembly;

FIG. 17 is a perspective view of a valve of the gas inlet connector;

FIG. 18 is a side view of the gas inlet connector;

FIG. 19 is a perspective view of the gas inlet connector;

FIG. 20 is a cross-sectional view of a gas inlet connector and insert of the ventilator manifold assembly;

FIG. 21 is a cross-sectional view showing the gas inlet connector of FIG. 20 with a valve thereof in an open state;

FIG. 22 is a cross-sectional view showing the gas inlet connector of FIG. 20 with the valve in a closed state;

FIG. 23 is a perspective view of an inline filtration element of the gas inlet connector of FIG. 20;

FIG. 24 is an exploded perspective view of the gas inlet connector and insert of FIG. 20;

FIG. 25 is an exploded perspective view of the insert and ventilator body;

FIG. 26 is a color version of FIG. 12; and

FIG. 27 is a color version of FIG. 13.

### DETAILED DESCRIPTION

The present disclosure encompasses various embodiments of a ventilator manifold assembly for use in ventilation therapy. The detailed description set forth below in connection with the appended drawings is intended as a description of several currently contemplated embodiments and is not intended to represent the only form in which the disclosed subject matter may be developed or utilized. The description sets forth the functions and features in connection with the illustrated embodiments. It is to be understood, however, that the same or equivalent functions may be accomplished by different embodiments that are also intended to be encompassed within the scope of the present disclosure. It is further understood that the use of relational terms such as first and second and the like are used solely to distinguish one from another entity without necessarily requiring or implying any actual such relationship or order between such entities.

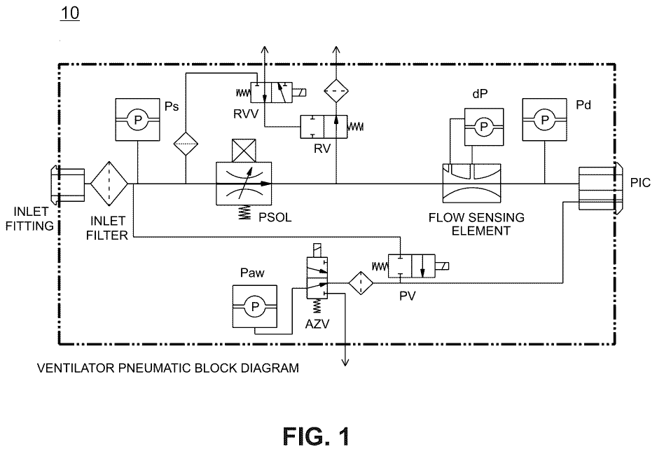

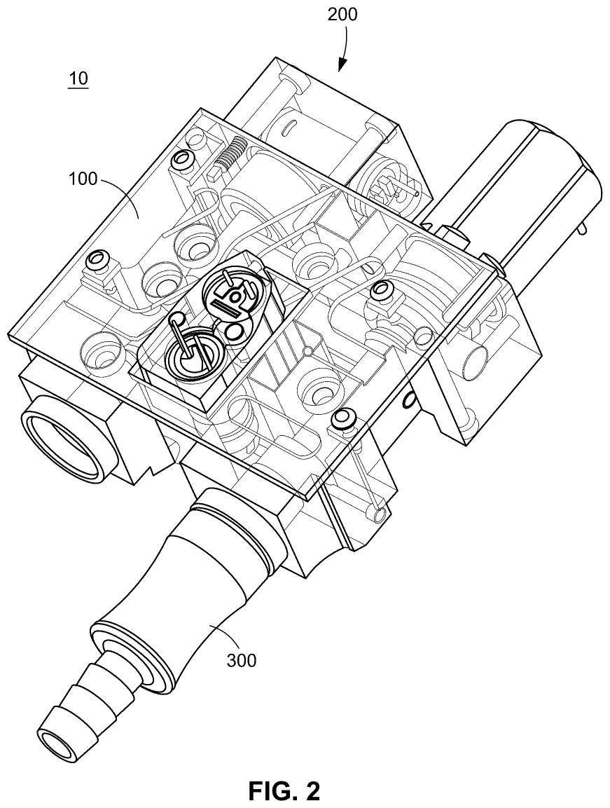

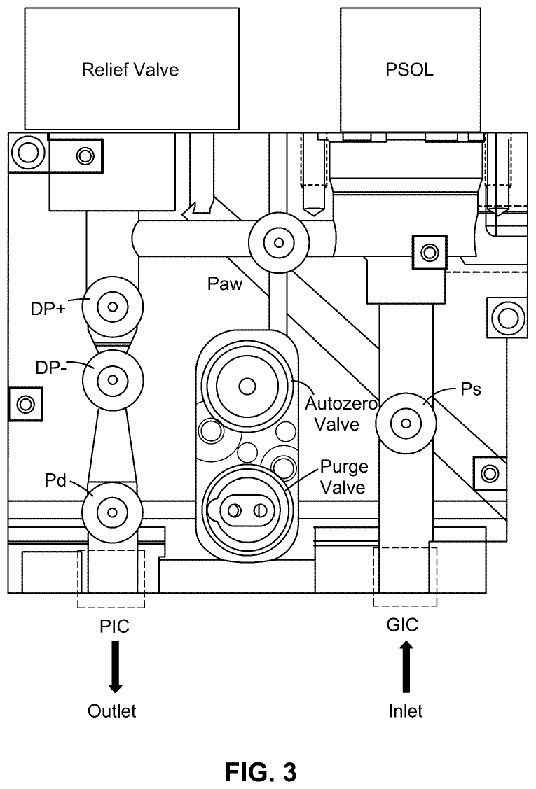

FIG. 1 is a schematic diagram of a ventilator manifold assembly 10 according to an embodiment of the present disclosure. FIG. 2 is a top perspective view of the ventilator manifold assembly 10 showing structural aspects of a ventilator manifold 100 thereof and related components of the ventilator manifold assembly 10 including a pressure relief valve (RV) 200 and gas inlet connector 300. FIG. 3 is a top view of the ventilator manifold assembly 10 showing a structural layout of various elements of the schematic diagram of FIG. 1. The ventilator manifold assembly 10 may receive a flow of gas including pressurized air and/or supplemental oxygen via the gas inlet connector (GIC) 300 (corresponding to the inlet fitting in FIG. 1) and provide the gas to a patient interface connector (PIC) via a proportional solenoid valve (PSOL) according to ventilation therapy parameters (e.g., gas volume, timing, etc.). Owing to structural aspects of the ventilator manifold assembly 10, including the manifold body 100, pressure relief valve (RV) 200, and gas inlet connector 300 thereof, the ventilator manifold assembly 10 may advantageously reduce the pressure drop associated therewith, while eliminating or minimizing the need for drying of accumulated moisture, increasing the performance and requiring less maintenance in comparison with conventional ventilators.

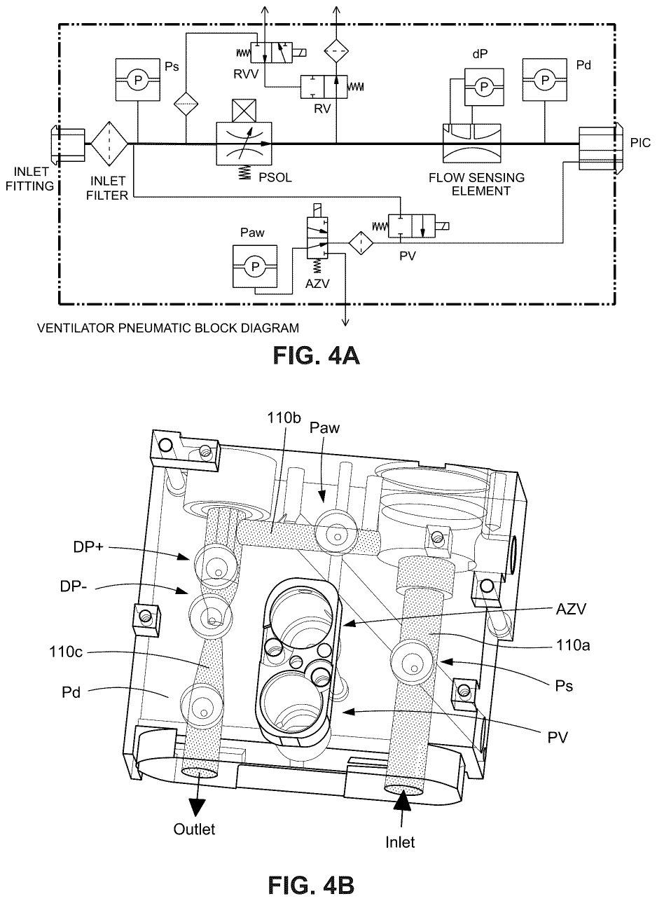

More specifically, as emphasized in FIGS. 4A and 4B (and with further reference to FIG. 3), the ventilator manifold assembly 10 may accept pressurized gas ranging from 8 to 25 psi from an external gas source via a gas hose connected to the inlet fitting (corresponding to the gas inlet connector 300 shown in FIG. 2), and the gas may be filtered by a 40-micron inlet filter to protect both the PSOL and the patient. After an inlet pressure sensor Ps measures the inlet pressure to detect a low or high inlet source pressure, the PSOL and downstream flow sensing element or flow sensor may be used to control the delivered flow of gas to the patient, with the PSOL delivering, for example, 0 to 40 LPM of flow and the flow sensor measuring the delivered flow from the PSOL. An electronic PI filter may use the output from the flow sensor to control the PSOL based on user settings. The flow sensor may have a fixed orifice flow element and a delta pressure sensor, dP (e.g., two pressure sensors DP+ and DP−), to measure the pressure drop across the flow element. The system may be calibrated during production to create a look-up table of dP versus flow. The piloted dump valve, RV (corresponding to the pressure relief valve 200 shown in FIG. 2), and a dump solenoid valve RVV are configured to form the safety system. The RVV default state may be open to atmosphere and energized during normal ventilation. The dump valve may therefore default to open so that all source gas will be dumped to atmosphere in the case when the PSOL is stuck open or on loss of power. This will prevent the patient from receiving excessive pressure in the case of a system failure. The RVV may be actively energized during normal ventilation, which in turns shuts the dump valve so that the gas from the PSOL goes to the patient.

Just downstream from the flow sensor is a delivered or driving pressure sensor, Pd. This sensor may be used to determine circuit disconnects and occlusions. Normal delivered pressure may vary from 0.5 to 16.6 psi based on the flow. The illustrated system also contains an airway pressure sensor, Paw, which may be used to measure the pressure in the patient's lungs. The patient circuit may have a patient pressure sense lumen that is connected to the Paw sensor via the patient interface connector, PIC. The Paw sensor may also have an autozero solenoid, AZV, to periodically zero the Paw sensor. In addition, there may be a purge solenoid valve, PV, which is used to periodically purge the pressure sense lumen in the patient circuit and keep the pressure line readings accurate. The autozero valve AZV may be located in between the purge and patient airway sensor Paw to keep the pressure sensor safe while purging. The patient interface connector, PIC, is used to connect the patient circuit to the ventilator. In the contemplated dual lumen system, one lumen may contain the delivered gas to the patient which comes from the PSOL, with the second lumen being used to measure the patient airway pressure via the Paw sensor.

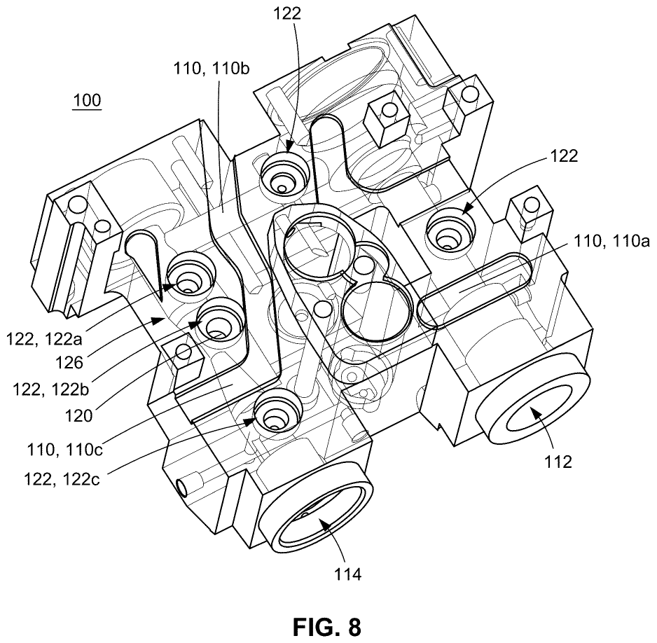

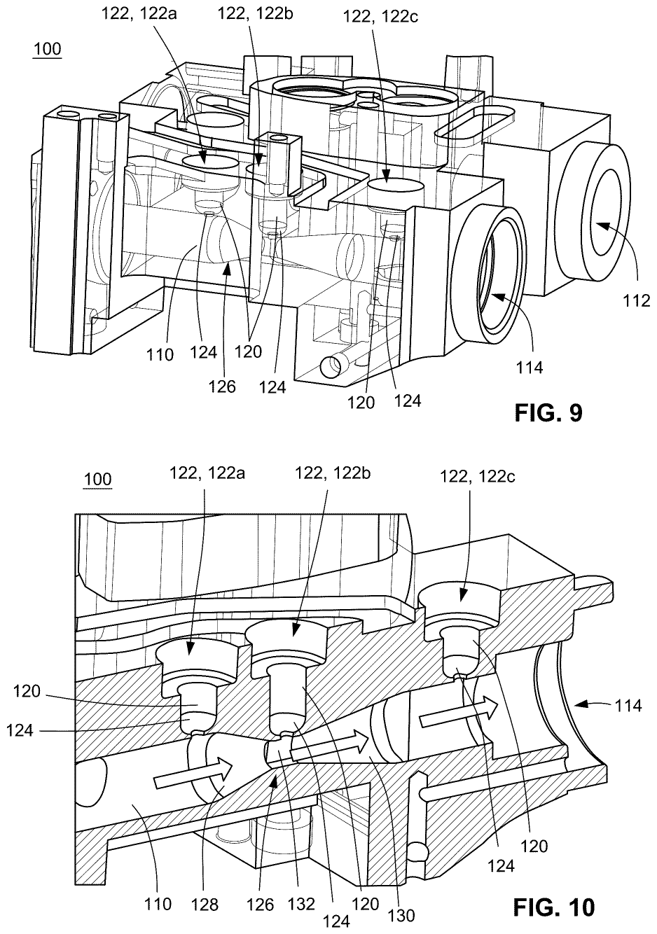

Referring to FIGS. 8-11, which show various views of the manifold body 100, the manifold body 100 may include a first interior region 110 defining a flow passage extending from a flow input 112 of the manifold body 100 that interfaces with the gas inlet connector 300 of the ventilator manifold assembly 10 (see also FIG. 2 and inlet fitting in FIG. 1) to a flow output 114 of the manifold body 100 that interfaces with the PIC (see also FIG. 1). As shown, the manifold body 100 may further include one or more second interior regions 120 respectively defining one or more pressure sense lines for providing fluid communication between the flow passage and corresponding pressure sense outputs 122 of the manifold body 100. The manifold body 100 may be molded from one or more pieces of material (e.g., a durable plastic) so as to have the first and second interior regions 110, 120 defining the flow passages, pressure sense lines, inlets, outlets, etc. described herein.

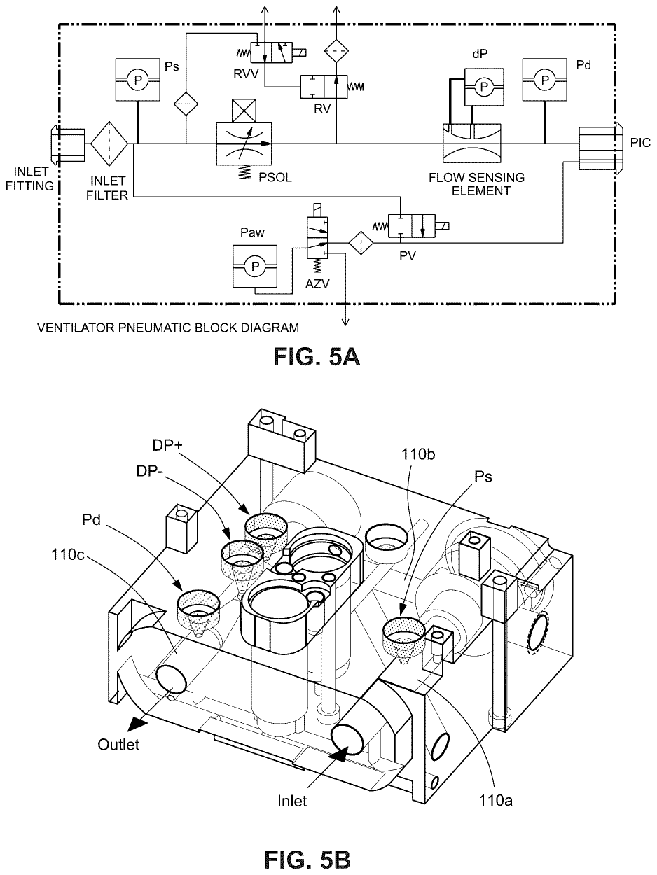

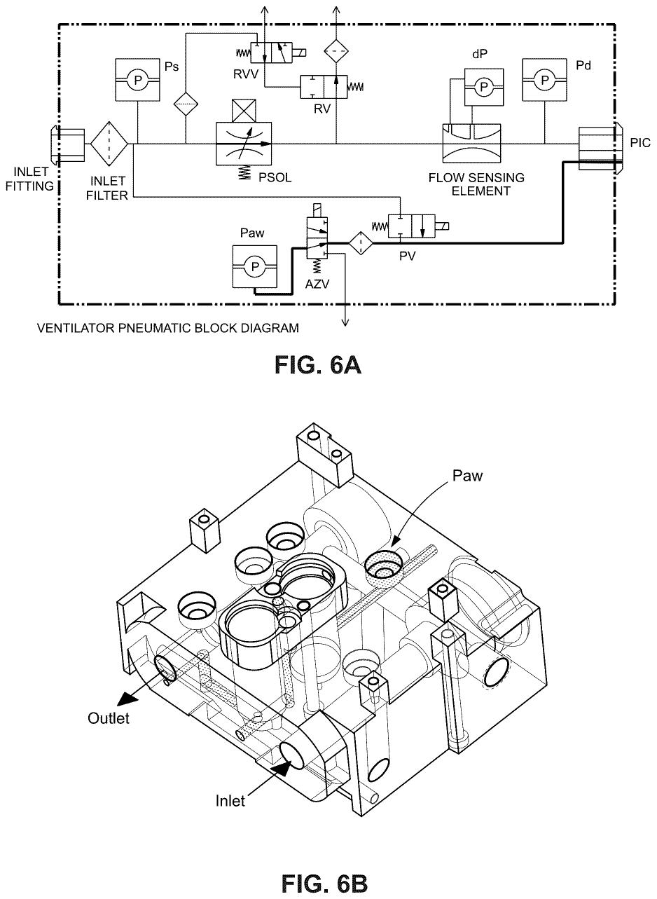

Referring to FIGS. 4A, 4B, and 8 together, the first interior region 110 of the manifold body 100 may comprise a first leg 110a defining the flow passage from the flow input 112 that interfaces with the gas inlet connector 300 (corresponding to the inlet fitting in FIG. 1) to the solenoid valve (PSOL), where a ninety-degree turn in the flow passage constitutes the end of the first leg 110a. The first interior region 110 of the manifold body 100 may further comprise a second leg 110b defining the flow passage from the solenoid valve (PSOL) to the location of the pressure relief valve (RV) 200 shown in FIG. 1 (described in more detail below), where another ninety-degree turn in the flow passage constitutes the end of the second leg 110b. The first interior region 110 of the manifold body 100 may further comprise a third leg 110c defining the flow passage from the pressure relief valve (RV) to the flow output 114 that interfaces with the PIC. As such, the flow passage collectively defined by the first, second, and third legs 110a, 110b, 110c may be shaped like a letter “U” with the flow input 112 and flow output 114 arranged in parallel on the same side of the manifold body 100. Along this flow passage, there may be various pressure sense lines by which pressure readings along the flow passage may be taken by pressure sensors Ps, Paw, dP, Pd as shown and described above in relation to FIG. 1, with additional reference to FIGS. 5A and 5B emphasizing the associated pressure sense lines. These may include, for example, a pressure sense line connecting the first leg 110a to the pressure sensor Ps, two pressure sense lines connecting the third leg 110c to the pressure sensor dP (as part of the flow sensing element shown in FIG. 1), and a pressure sense line connecting the third leg 110c to the pressure sensor Pd. Any or all of these pressure sense lines may be defined by a corresponding second interior region 120 of the manifold body 100 as described above and indicated in FIGS. 8-11, with each pressure sense line providing fluid communication between the flow passage defined by the first interior region 110 and a corresponding pressure sense output 122 of the manifold body 100. In particular, the pressure sense lines may provide fluid communication between the flow passage and the respective pressure sensor Ps, dP, Pd. As emphasized in FIGS. 6A and 6B, an additional pressure sense line may provide fluid communication between a pressure sense line in the PIC and the patient airway pressure sensor Paw (e.g., via the purge valve PV and autozero valve AZV).

In greater detail, as explained above in relation to the schematic representations in FIGS. 1, 5A, and 6A and with current reference to FIGS. 8-11, the flow sensor downstream of the PSOL may have a fixed orifice flow element 126 which is integrated into the third leg 110c. That flow element 126 comprises generally frusto-conical upstream and downstream regions 128, 130 which are fluidly coupled by an intervening orifice 132. Of the three pressure sense lines, and hence the three pressure sense outputs 122, which each fluidly communicate with the third leg 110c, a first pressure sense line with its corresponding first pressure sense output 122a is fluidly coupled to the third leg 110c just upstream of the upstream region 128 of the integrated flow element 126. A second pressure sense line with its corresponding second pressure sense output 122b is fluidly coupled to the orifice 132 of the flow element 126. Finally, a third pressure sense line with its corresponding third pressure sense output 122c is fluidly coupled to the third leg 110c downstream of the downstream region 130 of the flow element 126, in relative close proximity to the flow output 114. Those pressure sense lines including respective ones of the pressure sense outputs 122a, 122b are those which are in turn fluidly connected to the delta pressure sensor dP which, as indicated above, functions to measure the pressure drop across the flow element 126. That pressure sense line including the pressure sense output 122c is that which is in turn fluidly connected to the pressure sensor Pd to provide a measurement of the delivered pressure. In a similar vein, that pressure sense line which includes the sole pressure sense output 122 fluidly coupled to the first leg 110a is fluidly connected to the pressure sensor Ps (as represented in FIGS. 3, 4B, and 5B) to provide a measurement of the inlet pressure as also described above.

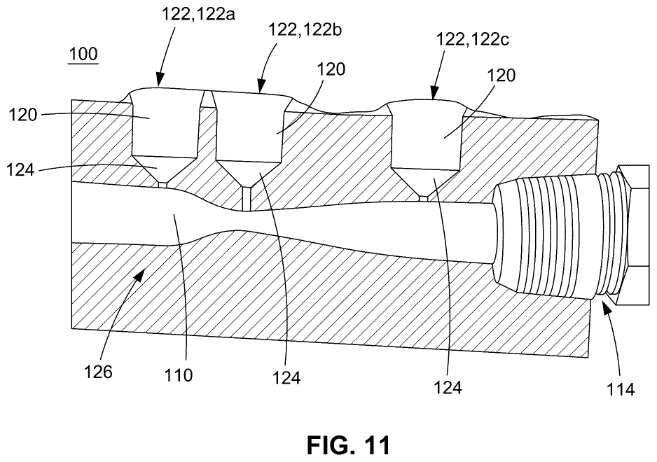

As depicted in FIGS. 8-11, each pressure sense line defined by a second interior region 120 of the manifold body 100 may have a first cross-sectional area at a position where the pressure sense line opens into the flow passage defined by the first interior region 110 and a second cross-sectional area greater than the first cross-sectional area at a position nearer to the pressure sense output 122 (and, thus, nearer to the pressure sensor). For example, as most clearly visible in FIG. 11, each pressure sense line may comprise a conical void 124 whose cross-sectional area increases in a direction from the flow passage defined by the first interior region 110 to the pressure sense output 122. The area of increased cross-sectional area may advantageously break the capillary action that would otherwise occur if the entire pressure sense line were a narrow passage, thus keeping any condensation or other moisture in the flow passage away from the pressure sensor. When any moisture gets past the narrow section (having the first cross-sectional area) of the disclosed pressure sense line, further movement along the pressure sense line causes the moisture to spray outward into the greater second cross-sectional area (e.g., into the conical void 124) instead of reaching the pressure sensor. Any moisture accumulated in the conical void 124 or other portion of the pressure sense line having the increased cross-sectional area may subsequently flow back into the flow passage rather than continuing on toward the pressure sensor as occurs in conventional pressure sense lines. As a result, any need to dry the pressure sense line may be completely eliminated or greatly minimized.

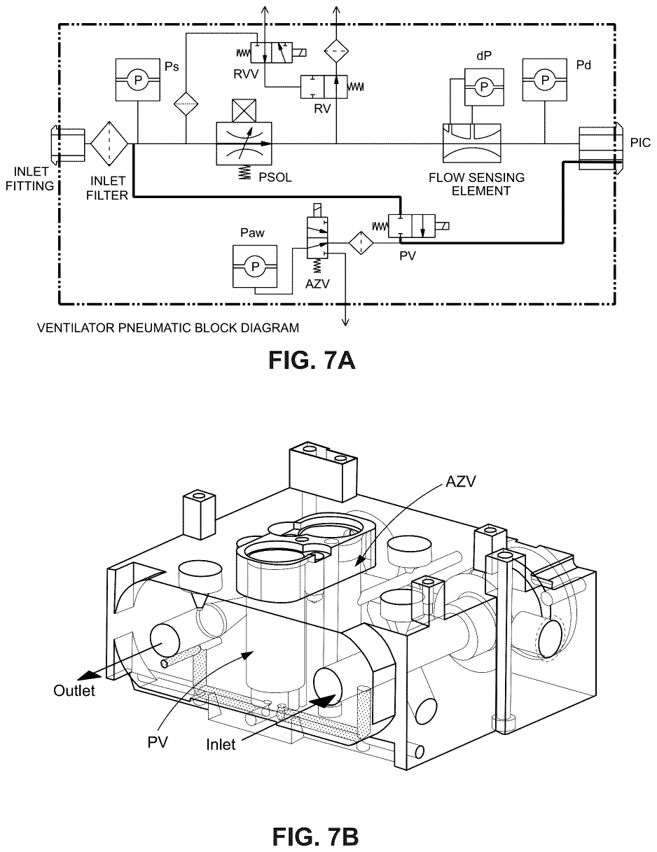

As noted above, there may be a purge solenoid valve, PV, which is used to periodically purge the pressure sense lumen in the patient circuit and keep the pressure line readings accurate. There may further be an autozero valve AZV located in between the purge and patient airway sensor Paw to keep the pressure sensor safe while purging. In more detail, with reference to FIGS. 6A, 6B, 7A, and 7B, the purge valve PV may periodically be actuated to connect the pressure sense lumen in the patient circuit to the high pressure upstream of the PSOL (see FIGS. 7A and 7B). This may purge any moisture that has accumulated in the pressure sense lumen (e.g., via a pressure tap in the patient interface). When the purge valve PV is actuated, the autozero valve AZV may simultaneously be actuated, thereby preventing the high purge pressure from damaging the sensitive patent airway sensor (Paw) while simultaneously venting the patient airway sensor (Paw) to atmosphere to zero the sensor.

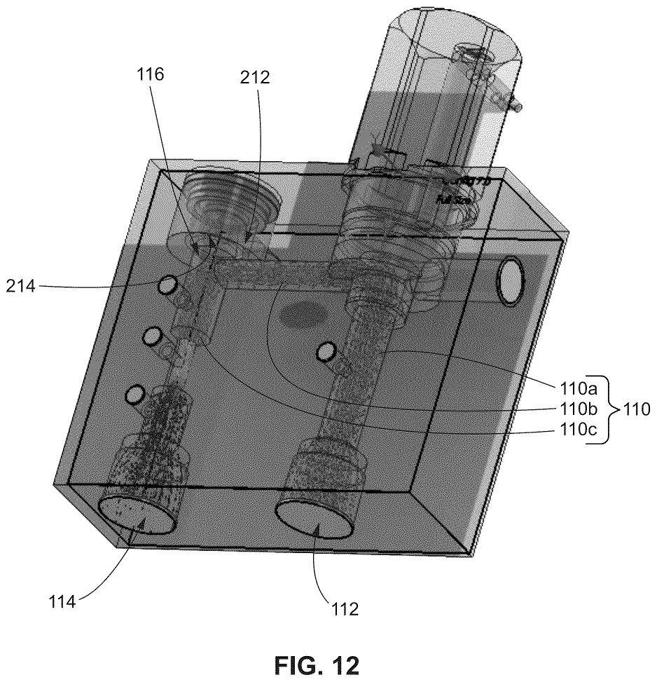

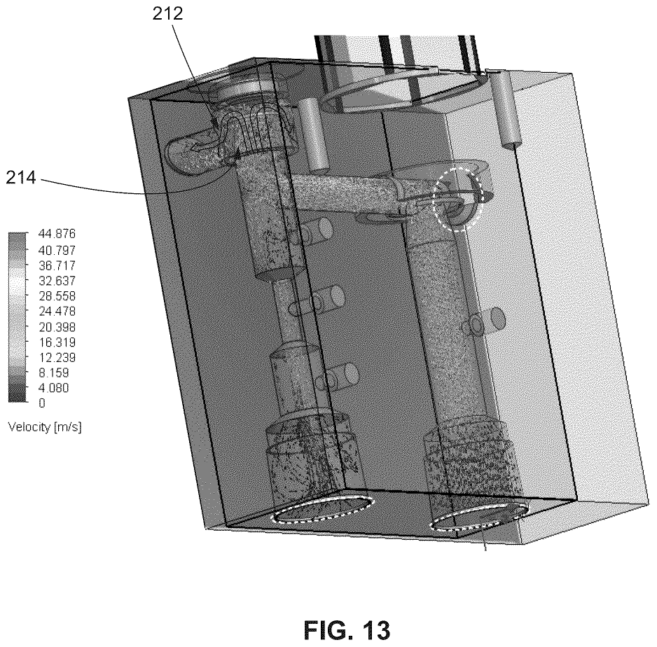

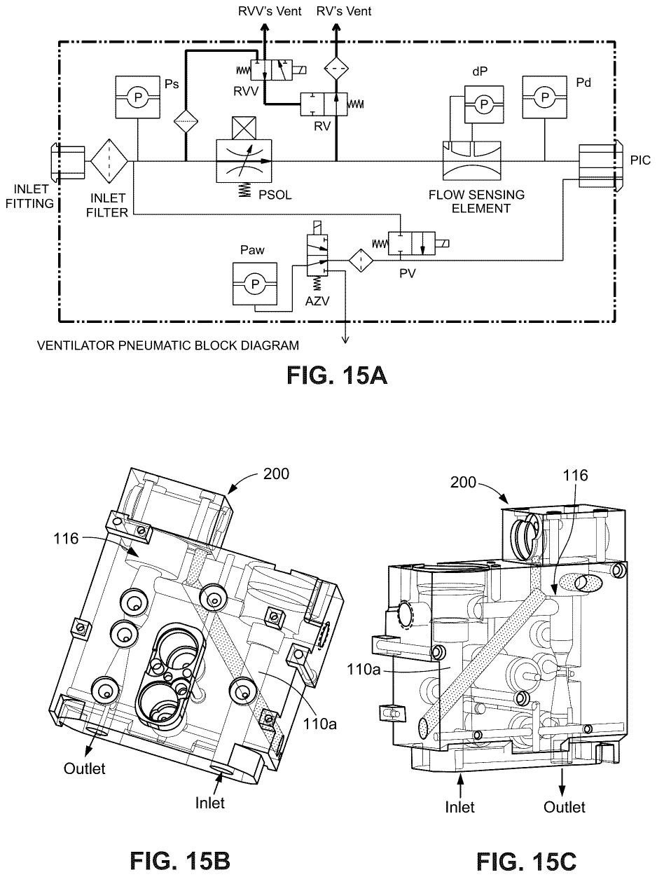

Referring to FIGS. 1, 2, and 12-14, and as emphasized in FIGS. 15A-15C, it is contemplated that the ventilator manifold assembly 10 may be designed to reduce the pressure drop that is conventionally associated with implementing a pressure relief valve. In this respect, the schematic views of FIGS. 1 and 15A depict the pressure relief valve 200 (indicated as “RV” in FIGS. 1 and 15A) as being arranged in parallel to (rather than in-line with) the flow passage. In particular, the first interior region 110 of the manifold body 100 that defines the flow passage may further define an opening 116 in the flow passage (see FIG. 12) between the flow input 112 and the flow output 114 of the manifold body 100, with the opening 116 being configured for arrangement of the pressure relief valve 200 in parallel to the flow passage. Preferably, the opening 116 in the flow passage may be located at a ninety-degree turn of the flow passage, such as at the intersection of the second and third legs 110b, 110c of the first interior region 110 defining the flow passage as shown in FIG. 12. The pressure relief valve 200 may be fluidly coupled to the opening 116 as shown in FIGS. 2, 15B, and 15C, for example, and may be configured to dump compressed gas from the flow passage. As can be seen in the simulation view of FIG. 12, the parallel connection to the closed pressure relief valve 200 causes no pressure drop in the system during normal operation of the ventilator, unlike conventional pressure relief valves that are provided in-line with the flow of gas and cause a pressure drop at all times. If the ventilator fails, the pressure relief valve 200 may be activated as shown in the simulation view of FIG. 13 to dump a portion of the compressed gas (e.g., more than 82%) out of the system and away from the patient's lungs.

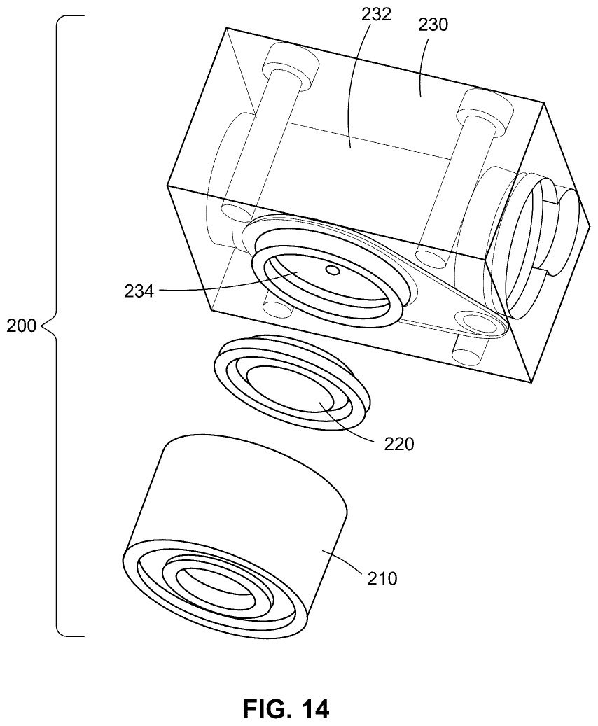

As shown in FIG. 14, the pressure relief valve 200 may include a seat 210 fluidly coupled to the opening 116 in the flow passage (see FIGS. 12, 15B, and 15C). The seat 210 may define a dump passage 212 for dumping the compressed gas. For example, the seat 210 may define a main passage 214 that is fluidly coupled to the opening 116 and spills into a dump passage 212 that is provided concentrically around the main passage 214 as shown in FIGS. 12 and 13. The pressure relief valve 200 may further include a diaphragm 220 configured to open and close the dump passage 212. For example, the diaphragm 220 may seat against the end of the main passage 214 to close the main passage 214 during normal operation. To this end, as shown in FIGS. 15A-15C, a dump solenoid valve RVV may actuate the pressure relief valve (RV) 200 by connecting the first leg 110a of the main flow passage of the ventilator 100 (i.e., upstream of the PSOL) to the side of the diaphragm 220 that is opposite the seat 210. For example, as shown in FIG. 14, the diaphragm 220 may be connected to a valve top 230 opposite the seat 210, with the valve top 230 defining a pressurization chamber 232 that is in fluid communication with the diaphragm 220 via an opening 234. When the dump solenoid valve RVV is actuated (e.g., electronically) during normal operation of the ventilator 100, the pressure taken from upstream of the PSOL pressurizes the pressurization chamber 232 and causes the diaphragm 220 to remain seated against the seat 210. Due to the pressure drop caused by the PSOL, this pressure will necessarily be greater than the pressure downstream of the PSOL acting on the other side of the diaphragm 220, ensuring that the pressure relief valve (RV) 200 will remain closed. When a fault is detected in the PSOL, the dump solenoid valve RVV will be de-actuated, causing the pressurization chamber 232 to vent to atmosphere (see FIG. 15A). The diaphragm 220 will then unseat in response to any accumulation of pressure downstream of the PSOL, such that the compressed gas that would otherwise go to the patient instead flows from the flow passage of the manifold body 100 through the main passage 214 and into the dump passage 212 (where it may be safely vented to atmosphere).

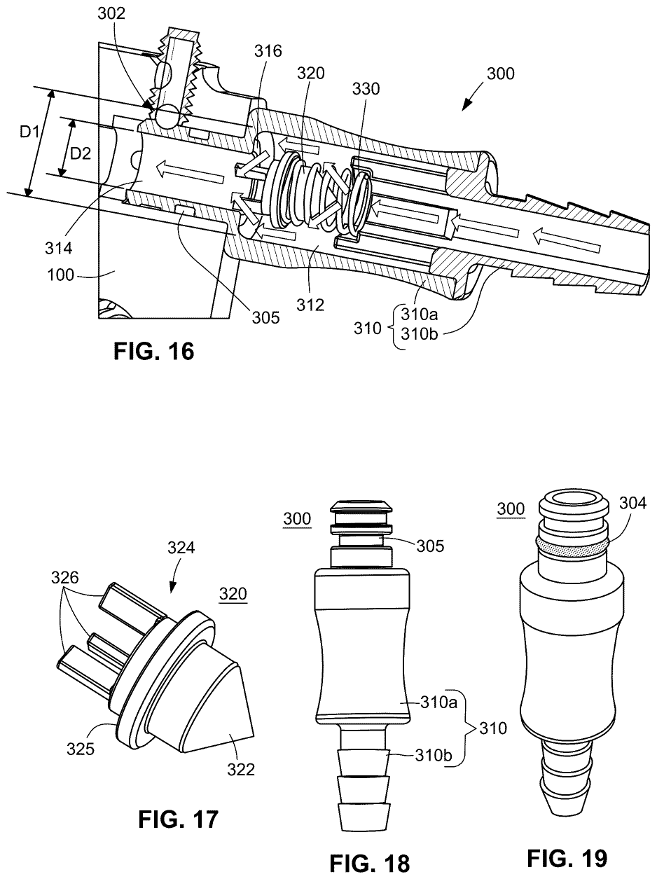

Referring to FIGS. 1, 2, and 16-25, it is contemplated that the ventilator manifold assembly 10 may be designed to reduce the pressure drop that is conventionally associated with a quick connect fitting or other connector for connecting source gas tubing from a gas source to the ventilator. In this respect, as schematically depicted as the inlet fitting in 1 and shown structurally in FIGS. 2 and 16-25, the ventilator manifold assembly 10 may include a gas inlet connector (GIC) 300, 300′ that minimizes pressure drop at a check valve 320, 320′ formed therein. In particular, as shown in FIG. 16, a gas inlet connector 300 may be connectable to the flow passage at the flow input 12 (see, e.g., FIGS. 2 and 8), where it may be releasably held in place by a detent mechanism 302 (e.g., ball) and sealed from the outside by an O-ring 304 (see FIG. 19) that may be fit into an O-ring slot 305 (see FIGS. 16 and 18). The gas inlet connector 300 may generally comprise a connector housing 310 defining a bore, with the connector housing 310 in some cases comprising a separate manifold connector piece 310a and hose fitting 310b.

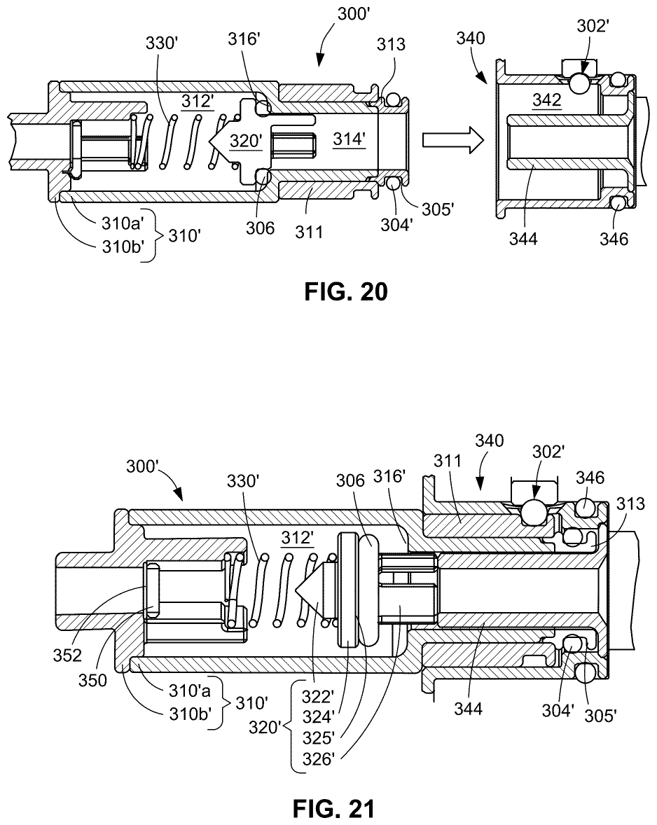

As shown in FIG. 16, the bore defined by the connector housing 310 may include a first portion 312 having a first diameter D1, a second portion 314 having a second diameter D2 less than the first diameter, and a shoulder 316 between the first and second portions 312, 314. The second portion 314 of the bore may correspond to the portion of the connector housing 310 that is inserted into the manifold body 100 as shown in the illustrated example. The gas inlet connector 300 may further include a valve 320 disposed within the bore 312, 314. The valve 320 may have a first end 322 that has a conical shape and a second end 324 that defines a surface 325 configured to seat against the shoulder 316 of the bore defined by the connector housing 310. A spring 330 within the bore may bias the valve 320 against the shoulder 316 to prevent gas flow while the gas inlet connector 300 is disconnected. When the gas inlet connector 300 is connected to the manifold body 100, a plunger formed in the manifold body 100 (not shown in FIG. 16 but described below in more detail in relation to FIGS. 20-25) may protrude from the manifold body 100 so as to hold the valve 320 away from the shoulder 316 to allow gas to flow through the gas inlet connector 300 and into the manifold body 100 in the direction of the arrows shown in FIG. 10. By virtue of the conical shape of the first end 322 of the valve 320, the valve 320 may advantageously present an aerodynamic surface to the oncoming gas, allowing the gas to easily deflect around the valve 320 in order to minimize the associated pressure drop.

Moreover, as best seen in FIG. 17, the valve 320 may have one or more alignment tabs 326 that protrude from the second end 324 of the valve 320 into the second portion 314 of the bore defined by the connector housing 310. The alignment tab(s) 326 may allow the valve 320 to remain in alignment with the second portion 314 of the bore while unseated from the shoulder 316 while imposing only a minimal impediment to gas flow, thus advantageously further reducing the pressure drop associated with the gas inlet connector 300. As shown in the illustrated example, each alignment tab 326 (of which there may preferably be three) may be radially configured, with each alignment tab 326 thus being oriented parallel to the gas flow direction as the gas flows around the second end 324 of the valve 320 from the first portion 312 of the bore to the second portion 314 of the bore. In this way, each alignment tab 326 may present as narrow a surface as possible to the oncoming gas while deriving structural integrity from its radial extent. The alignment tabs 326 may be evenly spaced in a circumferential direction of the second portion 314 of the bore. The resulting pressure drop associated with the gas inlet connector 300 may be less than 0.2 psi for example.

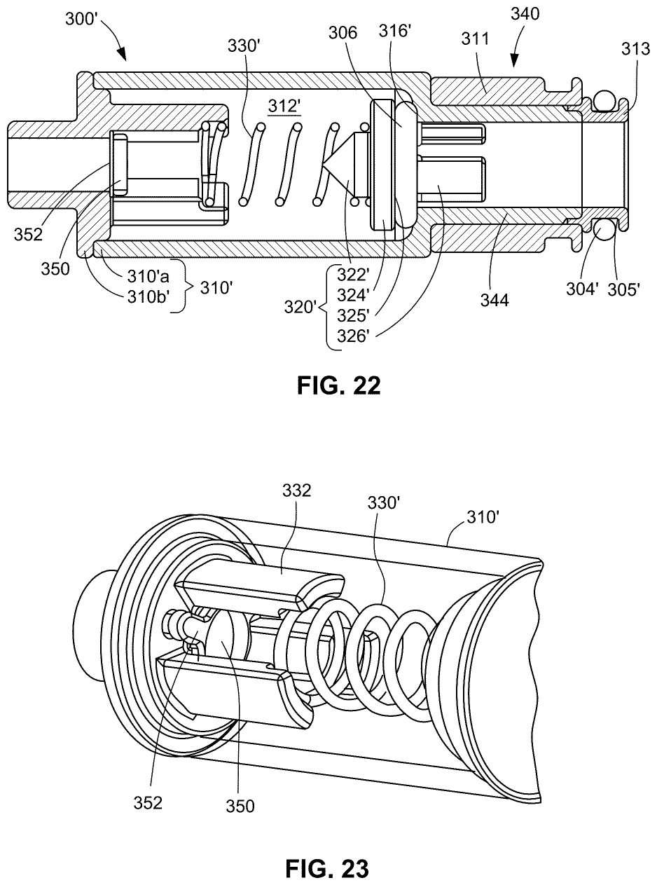

FIGS. 20-25 show another embodiment of a gas inlet connector 300′. The gas inlet connector 300′ may be the same as the gas inlet connector 300 except as described herein. In more detail, the gas inlet connector 300′ may comprise a connector housing 310′ (including manifold connector piece 310a′ and hose fitting 310b′ and defining first and second bore portions 312′, 314′ and shoulder 316′), a valve 320′ (including conical first end 322′, second end 324′, surface 325′ thereof, and alignment tabs 326′), and a spring 330′ that may be the same as the correspondingly numbered components of the gas inlet connector 300, except as described herein. Most significantly, referring to FIGS. 20 and 25, the gas inlet connector (GIC) 300′ may be used together with a GIC insert 340 that may be disposed within a borehole of the manifold body 100 to define the flow inlet 112. An O-ring 346 may be provided in a groove defined in the outside of the GIC insert 340 in order to seal the interior of the manifold body 100 from atmosphere. The interior of the GIC insert 340 may define a borehole 342 that serves as a receptacle for the GIC 300′. As shown in FIGS. 20, 21, and 25, the plunger 344 may be formed inside the borehole 342 as a protrusion arranged to unseat the valve 320′ as generally described above. FIGS. 21 and 22 respectively show the GIC 300′ inserted into the insert 340 and removed from the insert 340, and it can be seen in FIG. 21 that the plunger 344 may prevent the valve 320′ from closing by abutting the alignment tabs 326′ of the valve 320′, for example. As such, the alignment tabs 326′ may serve for plunger actuation in addition to alignment, as well as for venting (e.g., via the spaces between the alignment tabs 326′. The illustrated example of the GIC 300′ additionally includes an O-ring 306 that is provided on the surface 325′ to create a better seal with the shoulder 316′. It should be noted that the surface 325′ may be regarded as being configured to seat against the shoulder 316′ in the sense that is configured to seat against the shoulder 316′ via the O-ring 306. As shown in FIGS. 20, 21, and 24, the GIC 300′ may additionally comprise a detent ring 311 for engaging with the detent mechanism 302′ on the outside of the GIC 300′ (e.g., a catch for a ball detent), as well as a seal ring 313 that may define the groove 305′ for the O-ring 304′ (which may seal the interior of the insert 340 from atmosphere.

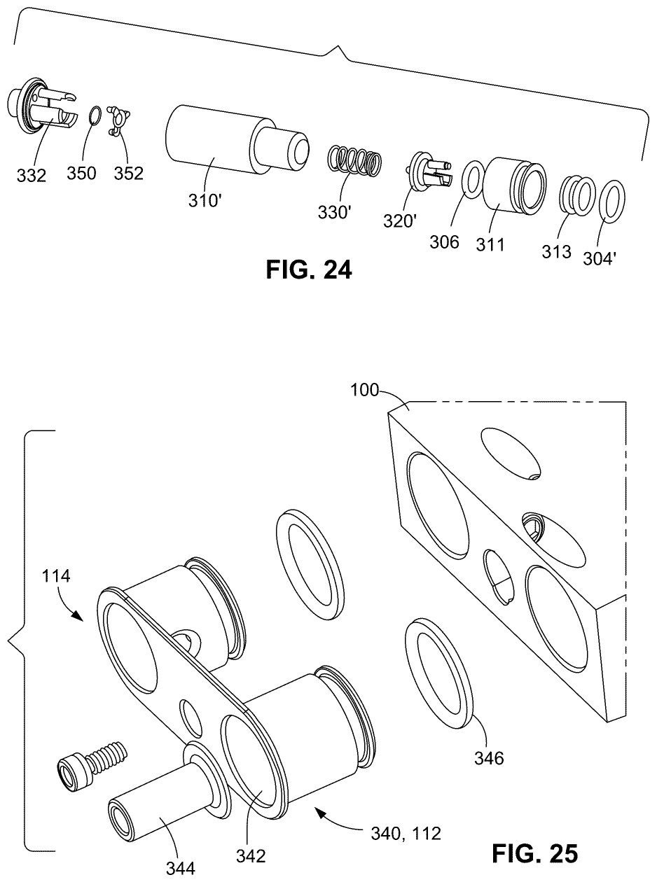

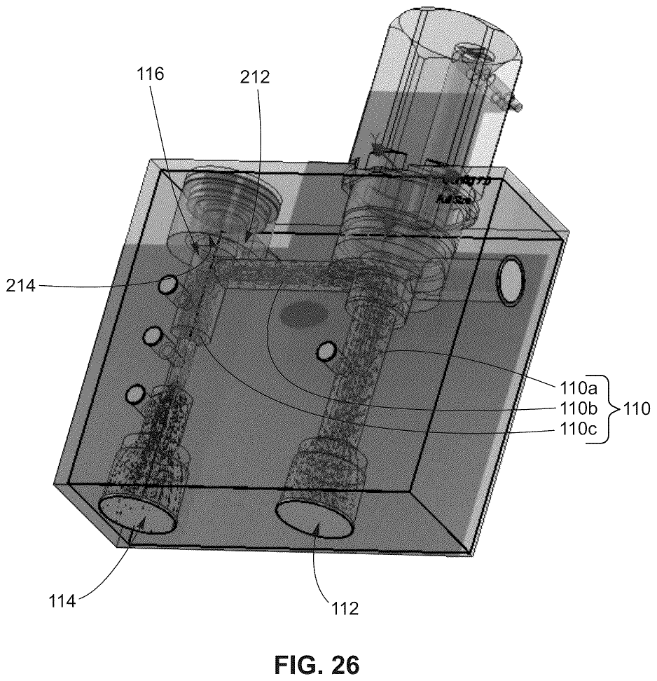

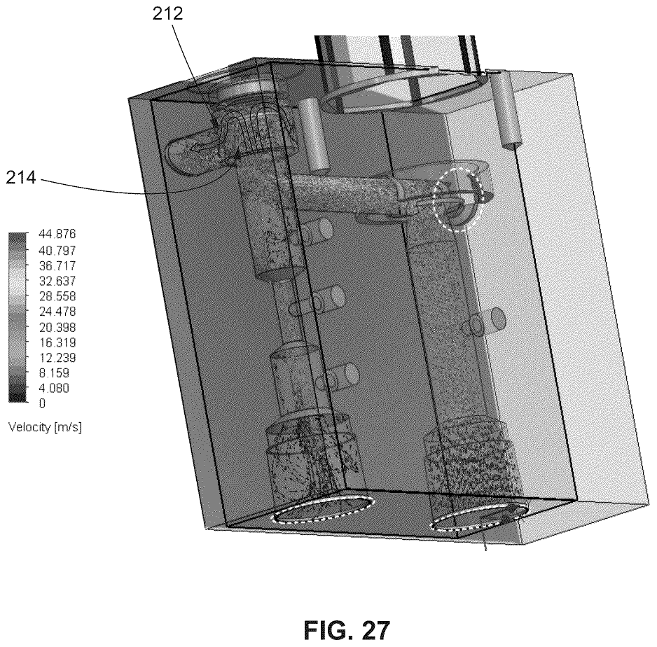

Referring to FIGS. 23 and 24, the GIC 300′ may further include an inline filtration element comprising a filter 350 and a filter retainer 352, which may be represented in the schematic views as the “inlet filter” (see FIG. 1). The filter 350 may be a 40-micron inlet filter, for example, and may serve to protect both the PSOL and the patient. The filter retainer 352 may centrally retain the filter 350 in the flow path of the gas as it enters the GIC 300′. As depicted in FIGS. 23 and 24, the filter 350 and filter retainer 352 may be surrounded by a plurality of spring retaining arms 332, which may be configured to collectively receive one end of the spring 330′ (the other end being in contact with the valve 320′). As illustrated, the spring retaining arms 323 may be disposed outside of the flow path of the gas such that the gas passing through the filter 350 may continue unimpeded toward the valve 320′, where the gas may then be deflected by the conical first end 322′ of the valve 320′ with minimal pressure drop as it passes through the GIC 300′. As shown in FIGS. 23 and 24, the filter retainer 352 may be shaped as a hub and spokes, with each spoke protruding between a pair of adjacent spring retaining arms 323.

The above description is given by way of example, and not limitation. Given the above disclosure, one skilled in the art could devise variations that are within the scope and spirit of the invention disclosed herein. Further, the various features of the embodiments disclosed herein can be used alone, or in varying combinations with each other and are not intended to be limited to the specific combination described herein. Thus, the scope of the claims is not to be limited by the illustrated embodiments.

What is claimed is:

1. A manifold body for a ventilator, the manifold body comprising:
a first interior region defining a flow passage extending from a flow input of the manifold body to a flow output of the manifold body; and
a second interior region defining a pressure sense line for providing fluid communication between the flow passage and a pressure sense output of the manifold body, the pressure sense line having a first cross-sectional area at a position where the pressure sense line opens into the flow passage and a second cross-sectional area greater than the first cross-sectional area at a position nearer to the pressure sense output.

2. The manifold body of claim 1, wherein the pressure sense line comprises a conical void whose cross-sectional area increases in a direction from the flow passage to the pressure sense output.

3. The manifold body of claim 1, wherein the first interior region further defines an opening in the flow passage between the flow input and the flow output of the manifold body, the opening being configured for arrangement of a pressure relief valve in parallel to the flow passage.

4. A ventilator comprising:
a flow passage for compressed gas; and
a pressure sense line for providing fluid communication between the flow passage and a pressure sensor, the pressure sense line having a first cross-sectional area at a position where the pressure sense line opens into the flow passage and a second cross-sectional area greater than the first cross-sectional area at a position nearer to the pressure sensor.

5. The ventilator of claim 4, wherein the pressure sense line comprises a conical void whose cross-sectional area increases in a direction from the flow passage to the pressure sensor.

6. The ventilator of claim 4, further comprising the pressure sensor.

7. The ventilator of claim 4, further comprising a pressure relief valve arranged in parallel to the flow passage and configured to dump compressed gas from the flow passage.

8. The ventilator of claim 4, further comprising a gas inlet connector connectable to the flow passage, the gas inlet connector including:
a connector housing defining a bore with a first portion having a first diameter, a second portion having a second diameter less than the first diameter, and a shoulder between the first and second portions;
a valve disposed within the bore, the valve having a first end that has a conical shape and a second end that defines a surface configured to seat against the shoulder, the valve further having one or more alignment tabs that protrude from the second end into the second portion of the bore; and
a spring biasing the valve toward the shoulder.

9. The ventilator of claim 4, further comprising:
a pressure sense passage connectable to a pressure sense lumen of a patient circuit; and
a second pressure sense line for providing fluid communication between the pressure sense passage and a second pressure sensor, the second pressure sense line having a first cross-sectional area at a position where the second pressure sense line opens into the pressure sense passage and a second cross-sectional area greater than the first cross-sectional area at a position nearer to the second pressure sensor.

10. The ventilator of claim 9, further comprising a purge valve operable to purge the pressure sense lumen of the patient circuit via the pressure sense passage.

11. The ventilator of claim 10, wherein the purge valve is operable to purge the pressure sense lumen by fluidly connecting the pressure sense lumen of the patient circuit to the flow passage via the pressure sense passage.

12. The ventilator of claim 11, further comprising an autozero valve operable to zero the second pressure sensor simultaneously with operation of the purge valve.

13. A ventilator comprising:
a flow passage for compressed gas; and
a pressure relief valve arranged in parallel to the flow passage and configured to dump compressed gas from the flow passage.

14. The ventilator of claim 13, wherein the pressure relief valve is fluidly coupled to an opening in the flow passage located at a ninety-degree turn of the flow passage.

15. The ventilator of claim 13, wherein the pressure relief valve includes:
a seat fluidly coupled to an opening in the flow passage and defining a dump passage for dumping the compressed gas; and
a diaphragm configured to open and close the dump passage.

16. The ventilator of claim 15, wherein the seat further defines a main passage that is fluidly coupled to the opening in the flow passage, the dump passage being defined concentrically around the main passage.

17. A ventilator comprising:
a flow passage for compressed gas; and
a gas inlet connector connectable to the flow passage, the gas inlet connector including:
a connector housing defining a bore with a first portion having a first diameter, a second portion having a second diameter less than the first diameter, and a shoulder between the first and second portions;
a valve disposed within the bore, the valve having a first end that has a conical shape and a second end that defines a surface configured to seat against the shoulder, the valve further having one or more alignment tabs that protrude from the second end into the second portion of the bore; and
a spring biasing the valve toward the shoulder.

18. The ventilator of claim 17, wherein the one or more alignment tabs comprise three radially configured alignment tabs.

19. The ventilator of claim 18, wherein the three radially configured alignment tabs are evenly spaced in a circumferential direction of the second portion of the bore.

20. The ventilator of claim 17, wherein the one or more alignment tabs are configured to abut a plunger disposed within the flow passage to unseat the valve.
